(19) United States
(12) Patent Application Publication
(10) Pub. No.: US 2009/0227666 A1
(43) Pub. Date: Sep. 10, 2009

(54) Enhancing Disease Resistance In An Animal

(75) Inventors: Dennis E. Jewell, Lawrence, KS (US); Steven C. Zicker, Lawrence, KS (US)

Correspondence Address: COLGATE-PALMOLIVE COMPANY, 909 RIVER ROAD, PISCATAWAY, NJ, 08855, US

(73) Assignee: Hill's Pet Nutrition, Inc., TOPEKA, KS (US)

(21) Appl. No.: 12/470,708

(22) Filed: May 22, 2009

Related U.S. Application Data
(62) Division of application No. 11/766,966, filed on Jun. 22, 2007.

(51) Int. Cl. A61K 31/385; A61K 31/355

(52) U.S. Cl. 514/440; 514/458

(57) ABSTRACT

A method to enhance disease resistance in an animal comprising feeding the animal a diet enriched in antioxidants and providing behavioral enrichment. In one embodiment, the behavioral enrichment comprises exercising the animal regularly effective to cause behavioral enrichment, wherein the feeding and exercising combination is effective to increase neutrophil phagocytosis and B cell percentages.

### FIELD OF THE INVENTION

This invention relates to methods for enhancing disease resistance in animals. Specifically, this invention relates to methods comprising a combination of feeding a diet enriched in antioxidants and providing behavioral enrichment to enhance disease resistance in animals.

### BACKGROUND OF THE INVENTION

Aging is caused in part by oxidants produced in the mitochondria as by-products of normal metabolism. Data indicate that in old rats, mitochondrial membrane potential, cardiolipin levels, respiratory control ratio, and overall cellular oxygen consumption are lower than in young rats, and the level of oxidants per unit oxygen and mutagenic aldehydes from lipid peroxidation are higher (B. N. Ames, Ann NY Acad Sci, 1019:406-411 (2004) and B. N. Ames, Arch Biochem Biophys, 423:227-234 (2004)). Studies indicate that feeding old rats the normal mitochondrial metabolites acetyl carnitine and lipoic acid for a few weeks restores mitochondrial function, lowers oxidants to the levels of young rats, increases ambulatory activity, and improves cognitive function test results. It is hypothesized that with aging there is increased oxidative damage to proteins and lipid membranes causing deformation of the structure of key enzymes with a consequent lessening of affinity for the enzyme substrate. Providing increased levels of substrate restores function. Thus, with aging, certain metabolites are considered necessary for health and, therefore, are conditional micronutrients.

Poorly maintained cellular redox levels lead to elevated activation of nuclear transcription factors, such as NF-κB. This transcription factor is responsible for a variety of extracellular signaling molecules involved in inflammation, tissue remodeling, oncogenesis and apoptosis. These processes are integral to many of the degenerative changes associated with aging (H. L. Hu et al., Mech Ageing Dev, 121:217-230 (2000)). NF-κB is also a key regulator of the inducible expression of many genes associated with immune function, e.g., transcriptional regulation of interleukin (IL)-1, interferon (IFN)-γ, IL-2. IL-6, and IL-8. In addition, inhibition of NF-κB has been suggested to be a major component of the anti-inflammatory activity of glucocorticoids, which are used for treatment of chronic inflammation (R. J. Farrell et al., J. Endocrinology, 178:339-346 (2003)). Based on this information, dietary antioxidants, by lowering oxidant levels, could play a significant role in reducing the inflammatory response.

In addition to decreasing oxidative stress, antioxidants such as vitamin E have also been shown to have anti-inflammatory properties. (See, G. Rimbach et al., Proc. Nutr. Soc., 61:415-425 (2002) and I. Jialal et al., Free Radic. Res., 36:1331-1336 (2002)). Vitamin E supplementation has been shown to decrease release of reactive oxygen species and lipid oxidation, to decrease release of cytokines such as IL-1 and tumor necrosis factor-alpha (TNF-α), and to decrease adhesion of monocytes to human endothelium. Vitamin E supplementation also decreases production of monocytic IL-6, plasma C-reactive protein (CRP), and soluble cell adhesion molecules such as intercellular adhesion molecule-1 (ICAM-1), vascular cell adhesion molecule-1 (VCAM-1), and E-selectin. The mechanism of inhibition of superoxide production and lipid oxidation by monocytes appears to be by the inhibition of protein kinase C, the inhibition of 5-lipoxygenase, which decreases IL-1β and TNF-α production, the inhibition of monocyte-endothelial cell adhesion via a decrease in adhesion molecules on monocytes (CD11b and VLA-4), and by decreasing DNA-binding activity of the transcription factor NF-κB. Long-term feeding of a vitamin E enriched diet to rats has been shown to improve the decline in cellular immune functions caused by aging, and appears to be associated with enhancement of both macrophage function and lymphocyte responsiveness (Sakai et al., J. Nutr. Sci. Vitaminology, 43:113-122 (1997)). It has also been demonstrated that increased vitamin E intake increases phagocytic ability of human neutrophils (Baehner et al., Ann. NY. Acad. Sci. 393:237-250 (1982); and De la Fuente, Eur. J. Clin. Nutr., 56:S5-S8 (2002)) and rat alveolar macrophages (Moriguchi et al., J. Nutr., 120:1096-1102 (1990)).

Vitamin C (ascorbic acid) has been used to treat clinical phagocytic cell dysfunctions (Hughes, Proc. Nutr. Soc., 58:79-84 (1999)), e.g., in Chediak-Higashi syndrome, which is characterized in part by defective neutrophil functions (Boxer et al., New Eng. J. Med., 295:1041-1045 (1976)).

Studies in recent years have shown that the antioxidant vitamins C and E improve phagocytic functions of neutrophils in humans, especially at advanced ages (Bergman et al., J. Nutr. Biochem., 15:45-50 (2004): Ventura et al., Cytobios., 77:225-232 (1994); Pallast et al., Am. J. Clin. Nutr., 69:1273-1281 (1999); and De la Fuente et al. Can. J. Physiol. Pharmacol., 76:373-380 (1998)). In addition, iodine uptake and nitroblue tetrazolium reduction by blood neutrophils is also improved in cows supplemented with B-carotene (Michal et al., J. Dairy Sci., 77:1408-1421 (1994)).

One of the most widely accepted theories proposed to explain aging is the free radical theory (De la Fuente, Eur. J. Clin. Nutr., 56:S5-S8 (2002)). According to this theory, oxygen-derived free radicals are responsible for the age-associated damage at the cellular and tissue levels through oxidative damage to biomolecules, with mitochondria being the main targets of free radical attack. This process is especially evident in the immune cells, which undergo changes that include enhanced, as well as diminished, functions. Protection of the immune cells by dietary antioxidant supplementation, especially in the elderly, to decrease morbidity and mortality is recommended. Indeed, oxidative damage appears to be an early and consistent change associated with the aging canine brain. Previously published data strongly supports the hypothesis that antioxidants, and in particular a broad spectrum of antioxidants, significantly reduces age-dependent cognitive dysfunction, which may be linked mechanistically to brain oxidative damage. (N. W. Milgram et al., Neurobiol. Aging, 26(1):77-90 (2005)). Vitamin E levels in serum are significantly higher and serum lipid peroxidation levels (malondialdehyde) are significantly lower in dogs receiving antioxidant-enriched food for 2.5 years. Significant improvements in landmark and oddity discrimination learning tasks are also observed in dogs receiving antioxidant-enriched food compared to controls. Dynamic contrast enhanced magnetic resonance imaging indicated that blood brain barrier permeability measures from a coronal section, including the hippocampus, increased at year 1 and year 2 in the control animals, but is maintained in the dogs receiving antioxidant-enriched food. It was concluded that a diet enriched with a broad spectrum of antioxidants can significantly improve cognitive function in aging dogs (Head et al. 8th International Conference on Alzheimer's Disease and Related Disorders. Jul. 20-25, 2002, Stockholm Sweden, Abstract published in Neurobiology of Aging, 23(1S):S115).

We have now discovered that dietary antioxidant enrichment in conjunction with behavioral enrichment increases neutrophil phagocytosis and B cell percentages. Thus, in a particular embodiment, the present invention is directed to a method to enhance disease resistance in animals comprising i) feeding animals a diet enriched in antioxidants and ii) exercising the animal effective to cause behavioral enrichment.

### BRIEF DESCRIPTION OF THE DRAWINGS

FIG. 1 is a bar graph showing the percentage of neutrophils that phagocytize Fluoresbrite® beads for each of four tested groups of dogs. “C-C” refers to data for canines receiving control food and control levels of behavioral enrichment. “A-C” refers to data from canines receiving antioxidant supplemented food and control levels of behavioral enrichment; “C-E” refers to data from canines receiving control food and enhanced levels of behavioral enrichment, “A-E” refers to data from animals receiving antioxidant enriched foods and enhanced levels of behavioral enrichment.

### SUMMARY OF THE INVENTION

We have identified the positive effect of enhancing disease resistance in animals by i) feeding a diet enriched in antioxidants and ii) providing behavioral enrichment. Accordingly, the present invention is directed to a method to enhance disease resistance in an animal comprising feeding the animal a diet enriched in antioxidants and providing behavioral enrichment to the animal. Behavioral enrichment may comprise, e.g., exercising the animal regularly effective to cause behavioral enrichment, wherein the feeding and exercising combination is effective to increase neutrophil phagocytosis and B cell percentages in said animal.

The present invention is also directed to a kit comprising an animal feed composition enriched in antioxidants; and instructions to enhance disease resistance in the animal fed the animal feed composition by providing behavioral enrichment, including but not limited to, exercising the animal regularly effective to cause behavioral enrichment.

Thus, in one aspect, the present invention is directed to a method to enhance disease resistance in an animal comprising feeding the animal a diet enriched in antioxidants; and providing behavioral enrichment to the animal. In one embodiment, enhanced disease resistance comprises an increase in neutrophil phagocytosis and B cell percentages in said animal.

Thus, in an embodiment, the present invention is directed to a method to enhance disease resistance in an animal comprising a) feeding the animal a diet enriched in antioxidants; and b) providing behavioral enrichment to the animal; wherein the feeding and providing behavioral enrichment combination is effective to increase neutrophil phagocytosis and B cell percentages in the animal.

In an embodiment of this aspect, the method comprises a diet that comprises d1-alpha-tocopherol acetate at about 1000 ppm or more.

In another embodiment of this aspect, the method comprises a diet that comprises 1-carnitine at about 275 ppm or more.

In still another embodiment of this aspect, the method comprises a diet that comprises d1-alpha-lipoic acid at about 125 ppm or more.

In another embodiment of this aspect, the method comprises a diet that comprises ascorbic acid at about 80 ppm or more.

In yet another embodiment of this aspect, the method comprises a diet that comprises about 1% of each of any one or more ingredients chosen from spinach flakes, tomato pomace, grape pomace, carrot granules, or citrus pulp.

In one embodiment, the behavioral enrichment comprises exercising the animal regularly effective to cause behavioral enrichment.

In a further embodiment, the exercising may comprise hand walking said animal for at least 30 minutes twice a week

In yet still another embodiment of this aspect, the method comprises behavioral enrichment that comprises providing animal to animal interaction and/or providing the animal with toys as a means to provide environmental and/or cognitive enrichment.

In a second aspect, the present invention is directed to a kit comprising an animal feed composition enriched in antioxidants; and instructions to enhance disease resistance in an animal fed the animal feed composition by providing behavioral enrichment.

In an embodiment of this second aspect, the animal feed composition comprises d1-alpha-tocopherol acetate at about 1000 ppm or more.

In still another embodiment of this second aspect, the animal feed composition comprises 1-carnitine at about 275 ppm or more.

In yet still another embodiment of this second aspect, the animal feed composition comprises d1-alpha-lipoic acid at about 125 ppm or more.

In still another embodiment of this second aspect, the animal feed composition comprises ascorbic acid at about 80 ppm or more.

In yet another embodiment of this aspect, the diet comprises about 1% each of any one or more ingredients chosen from spinach flakes, tomato pomace, grape pomace, carrot granules, or citrus pulp.

In one embodiment, the behavioral enrichment comprises exercising the animal regularly effective to cause behavioral enrichment.

In a further embodiment, the exercising may comprise hand walking said animal for at least 30 minutes twice a week.

In yet still another embodiment of this aspect, the method comprises behavioral enrichment that comprises providing animal to animal interaction and/or providing the animal with toys as a means to provide environmental and/or cognitive enrichment.

The instant application also relates to the use of a diet enriched in antioxidants and behavioral enrichment to enhance disease resistance in an animal.

In another aspect, the present invention is directed to a kit comprising a) an animal feed composition enriched in antioxidants; and b) instructions to enhance disease resistance in the animal fed the animal feed composition by providing behavioral enrichment to the animal: wherein the combination of feeding the feed composition and providing behavioral enrichment to said animal is effective to increase neutrophil phagocytosis and B cell percentages in the animal.

In still another aspect, a method to enhance disease resistance in an animal comprises: a) providing an animal feed composition enriched in antioxidants: and b) providing instructions to enhance disease resistance in the animal fed the animal feed composition by providing behavioral enrichment to the animal; wherein the combination of feeding the feed composition and providing behavioral enrichment to the animal is effective to increase neutrophil phagocytosis and B cell percentages in the animal.

Additional or alternative advantages and benefits of the present invention will be apparent to one of skill in the art.

### DETAILED DESCRIPTION OF THE INVENTION

Unless defined otherwise, all technical and scientific terms used herein have the same meanings as commonly understood by one of ordinary skill in the art to which this invention belongs. All patents, patent applications, publications, and other references cited or referred to herein are incorporated by reference. However, where there is a conflict between a definition in the present disclosure and that of a cited reference, the present disclosure controls.

As used herein, ranges are a shorthand for describing each and every value within a range, including endpoints.

As used herein and in the appended claims, the singular forms “a”, “an”, and “the” include plural reference unless the context clearly dictates otherwise.

As used herein, “an amount effective”, “an effective amount”, and like terms refer to that amount of a compound, material, activity or composition as described herein that may be effective to achieve a particular biological result. Such results may include, but are not limited to, the enhancement of immune function in an animal. Such effective activity may be achieved, for example, by administration of compositions of the present invention to an animal according to the methods disclosed herein. An effective amount may be based on several factors, including an animal's ideal weight, the metabolizable energy of the composition, and frequency of feeding the animal the compositions of the present invention, e.g. once, twice, or three times daily, and other compositions fed to the animal.

As used herein, an “effective amount of time” may be determined by observing or measuring a desired aspect in an animals behavior or body functions, e.g., an enhancement in immune response and/or disease resistance in an animal, and may be determined by one of skill in the art without undue experimentation.

A used herein, a method to “enhance disease resistance” includes enhancing the immune response of an animal. This includes increasing neutrophil phagocytosis as well as causing an increase in B cell percentages in an animal.

As used herein, “behavioral enrichment” includes, but is not limited to, subjecting an animal to one or more series of activities that can provide mental and/or physical stimulation of the animal that is beneficial to the well being of the animal, mentally or physically. Such activities may include exercise of any type, (e.g. running or playing with kennel mates or human companions, hand walking by leash for a period of time, e.g., 30 minutes or longer several limes a week). Appropriate amounts and types of exercise may be utilized such that behavioral enrichment is provided. This behavioral enrichment involves aerobic metabolism resulting in brain enhancement through decisions made during exercise. These decisions include both the cognitive processes inherent in the exercise (i.e. balance and locomotion) as well as the decisions such as response to commands or companionship.

The term “behavioral enrichment” also encompasses environmental enrichment (e.g., providing the animal with new toys which engage the animal's mental faculties) as well as a programs or activities that provide cognitive enrichment. Cognitive enrichment for an animal may include, but is not limited to, e.g., oddity determination learning problems or games of cognition. Such games may be played using food as a reward. Such games require successful cognitive activity to achieve the reward. Additional appropriate types of behavioral enrichment for an animal are familiar to one of skill in the art.

As used herein, “exercising an animal regularly sufficient to cause behavioral enrichment” and like terms refers to any form of exercise or activity that an animal may experience that requires a choice be made by the animal as well as changing the environment to cause the animal to have a mental response requiring learning. Examples of regular exercise sufficient to cause behavioral enrichment includes, but is not limited to, hand walking of an animal (e.g., a dog) for 30 minutes, twice a week.

The present invention relates to any animal, preferably a mammal, more preferably a companion animal. The term “companion animal” refers to any animal that lives in close association with humans and includes, but is not limited to, canines and felines of any breed. For example, it is contemplated herein that this term may also encompass any animal whose diet may be controlled by humans and which may benefit from the methods of the present invention. These animals may include, for example, domesticated farm animals (e.g. cattle, horses, swine, etc.) as well as undomesticated animals held in captivity, e.g. in zoological parks and the like. Typically, companion animals are cats and dogs.

All percentages expressed herein are on a weight by dry matter basis unless specifically stated otherwise.

Without being limited to any theory or particular mode of action, the present invention is based on the discovery of a method to enhance disease resistance and/or immune function in an animal comprising i) feeding the animal a diet enriched in antioxidants and ii) providing behavioral enrichment; wherein the combination is effective to increase neutrophil phagocytosis and B cell percentages in said animal.

As contemplated herein, a composition for use in the methods of the present invention is a diet enriched in antioxidants. As used herein, a diet or feed composition “enriched in antioxidants” refers to a diet that contains higher levels of antioxidants than typically found in an animal diet, for example, the antioxidant enriched diet disclosed in U.S. Pat. No. 6,914,071.

An antioxidant is a material that quenches a free radical. Examples of such materials include foods such as Ginkgo Biloba, citrus pulp, grape pomace, tomato pomace, carrot and spinach, as well as various other materials such as beta-carotene, selenium, coenzyme Q10 (ubiquinone), lutein, tocotrienols, soy isoflavones. S-adenosylmethionine, glutathione, taurine. N-acetylcysteine, vitamin E, vitamin C, alpha-lipoic acid, 1-carnitine and the like. Vitamin E can be administered as a tocopherol or a mixture of tocopherols and various derivatives thereof such as esters like vitamin E acetate, succinate, palmitate, and the like. The alpha form is preferable but beta, gamma and delta forms can be included. The d form is preferable but racemic mixtures are acceptable. The forms and derivatives will function in a vitamin E like activity after ingestion by the pet. Vitamin C can be administered in this diet as ascorbic acid and its various derivatives thereof such as calcium phosphate salts, cholesteryl salt, 2-monophosphate, and the like which will function in a vitamin C like activity after ingestion by the pet. They can be in any form such as liquid, semisolid, solid and heat stable form. Alpha-lipoic acid can be administered into the diet as alpha lipoic acid or as a lipoate derivative as in U.S. Pat. No. 5,621,117, racemic mixtures, salts, esters or amides thereof. L-carnitine can be administered in the diet and various derivatives of carnitine such as the salts such as the hydrochloride, fumarate and succinates, as well as acetylated carnitine, and the like can be used.

The quantities administered in the diet, all as wt % (dry matter basis) of the diet, are calculated as the active material, per se, that is measured as free material. The maximum amounts employed should not bring about toxicity. For example, at least about 100 ppm or at least about 150 ppm of vitamin E can be used. A range of about 500 to about 1,000 ppm can be employed, but useful levels are also 1000 ppm or more. A maximum of about 2000 ppm or about 1500 ppm is generally not exceeded. With respect to vitamin C at least about 50 ppm, 80 ppm and 100 ppm or more may be used. The quantity of d1-alpha-lipoic acid can vary from at least about 25 ppm, 50 ppm, 125 ppm or more may be used. Maximum quantities can vary about 100 ppm to 600 ppm or to an amount which remains non toxic to the pet. A possible useful range is about 100 ppm to about 200 ppm. For 1-carnitine about 50 ppm, 200 ppm, 275 ppm or more for canines are a useful minimum. For felines, slightly higher minimums of 1-carnitine can be employed such as about 100 ppm, 200 ppm, and 500 ppm. A nontoxic maximum quantity can be employed, for example, less than about 5,000 ppm. For canines, lower quantities can be employed, for example, less than about 5,000 ppm. For canines a useful range is about 200 ppm to about 400 ppm. For felines a useful range is about 400 ppm to about 600 ppm. Beta-carotene at about 1-15 ppm can be employed. Selenium at about 0.1 up to about 5 ppm can be employed. Lutein at least about 5 ppm can be employed. Tocotrienols at least at about 25 ppm can be employed. Coenzyme Q10 at least at about 25 ppm can be employed. S-adenosylmethionine at least at about 50 ppm can be employed. Taurine at least at about 1000 ppm can be employed. Soy isoflavones at least at about 25 ppm can be used. N-acetylcysteine at least at about 50 ppm can be used. Glutathione at least at about 50 ppm can be used. Gingko biloba at least at 50 ppm of extract can be used.

Spinach pomace, tomato pomace, citrus pulp, grape pomace, carrot granules, broccoli, green tea, ginkgo biloba and corn gluten meal are examples of substances that are high in oxygen radical absorbing capacity (ORAC). When added to the diet, e.g., as 1% inclusions (for a total of 5% substitution for a low ORAC ingredient such as corn) they increase the ORAC content of the overall diet and can increase the ORAC content of the plasma of animals which eat a diet containing these components. Preferably, any ingredient with an enhanced ORAC content could be used. Ideally, added at 1% combination with four other 1% ingredients for a total of 5% addition to the diet.

The compositions for use in the methods of the present invention, besides being enriched in antioxidants, also contain mitochondrial cofactors. “Mitochondrial cofactors” refers to any substance that may be useful to increase mitochondrial function. Such substances include, but are not limited to, antioxidants, electron transfer mediators, and enzyme cofactors such as, e.g., ubiquinone, antioxidants such as ascorbic acid, vitamin E, and lipoic acid; riboflavin; thiamin; niacin; vitamin K (phylloquinone and menadione); creatine; and carnitine.

Mitochondrial cofactors often include antioxidants but also may include other substances such as, electron transfer mediators, and enzyme cofactors such as, e.g., ubiquinone, antioxidants such as ascorbic acid or other forms of vitamin C, vitamin E, and lipoic acid; riboflavin; thiamin; niacin; vitamin K (phylloquinone and menadione); creatine; and carnitine. Effective amounts of mitochondrial cofactors for inclusion in the antioxidant enriched compositions administered in the methods disclosed herein may be shown by decreasing oxidative stress markers (such as alkenals). Mitochondria control oxidative stress markers because they are the source of most of the free radicals which provide cellular oxidative stress. Effective change in this marker of mitochondrial health would be a 20% reduction in circulating alkenal concentration. Some examples of dietary concentrations of mitochondrial cofactors are vitamin E between 400 and 2000 IU/kg, lipoic acid between 50 and 500 mg/kg and ascorbic acid between 50 and 500 mg/kg.

It is contemplated herein that the antioxidant enriched pet foods for use is in the methods of the present invention are nutritionally complete and balanced pet food compositions. Nutritionally complete and balanced pet food compositions are familiar to one of skill in the art. For example, nutrients and ingredients such as those disclosed herein as well as others suitable for animal feed compositions, and recommended amounts thereof, may be found, for example, in the Official Publication of the Associate of American Feed Control Officials, Inc. (“AAFCO”), Nutrient Requirements of Dogs and Cats, 2006.

Protein may be supplied by any of a variety of sources known by those skilled in the art, including plant sources, animal sources, or both. Animal sources include, for example, meat, meat by-products, seafood, dairy, eggs, etc. Meats include, for example, the flesh of poultry, fish, and mammals (e.g., cattle, pigs, sheep, goats, and the like). Meat by-products include, for example, lungs, kidneys, brain, livers, and stomachs and intestines (freed of all or essentially all their contents). The protein can be intact, almost completely hydrolyzed, or partially hydrolyzed. Protein content of foods may be determined by any number of methods known by those of skill in the art, for example, as published by the Association of Official Analytical Chemists in Official Methods of Analysis (“OMA”). The amount of “crude protein” in a composition disclosed herein may be determined based on the amount of nitrogen in the composition according to methods familiar to one of skill in the art.

Fat can be supplied by any of a variety of sources known by those skilled in the art, including meat, meat by-products, fish oil, and plants. Plant fat sources include wheat, flaxseed, rye, barley, rice, sorghum, corn, oats, millet, wheat germ, corn germ, soybeans, peanuts, and cottonseed, as well as oils derived from these and other plant fat sources. Fat content of foods may be determined by any number of methods known by those of skill in the art, for example, as published by OMA.

Carbohydrate may be supplied by any of a variety of sources known by those skilled in the art, including oat fiber, cellulose, peanut hulls, beet pulp, parboiled rice, corn starch, corn gluten meal, and any combination of those sources. Grains supplying carbohydrate include, but are not limited to wheat, corn, barley, and rice. Carbohydrate content of foods may be determined by any number of methods known by those of skill in the art. Generally, carbohydrate percentage may be calculated as nitrogen free extract (“NFE”), which may be calculated as follows: NFE=100%−moisture %−protein %−fat %−ash %−crude fiber %.

Dietary fiber refers to components of a plant which are resistant to digestion by an animal's digestive enzymes. Dietary fiber components of foods may be determined by any number of methods known by those of skill in the art, for example, as published by OMA. Dietary fiber includes soluble and insoluble fibers.

Soluble fiber are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fiber may be supplied by any of a variety of sources, including cellulose, whole wheat products, wheat oat, corn bran, flax seed, grapes, celery, green beans, cauliflower, potato skins, fruit skins, vegetable skins, peanut hulls, and soy fiber. Soluble and insoluble fiber content of foods may be determined by any number of methods known by those of skill in the art, for example, as published by OMA.

Crude fiber includes indigestible components contained in cell walls and cell contents of plants such as grains, e.g., hulls of grains such as rice, corn, and beans. Crude fiber content of foods may be determined by any number of methods known by those of skill in the art, for example, as published by OMA.

If desired, the amino acid percentage of the compositions in the present invention may be determined by any means known in the art. The values for the total amount of lysine provided by the invention can be determined using methods known in the art, for example, as published by OMA. Further, tryptophan content, as well as methionine, cysteine and other amino acid content may be determined according to methods known in the art, for example, as published by OMA. Amino acid content may also be determined according to methods known in the art, for example, as published by OMA. The essential amino acids in the present compositions may be supplied by any number of sources, including crude protein, or addition of free amino acids to the composition.

Metabolizable energy (ME) of a diet is the energy available to an animal upon consumption of the diet after subtracting the energy excreted in feces, urine, and combustible gases. Metabolizable energy values may be determined by methods known by those skilled in the art, such as detailed in Association of American Feed Control Officials: Official Publication, pages 160-165 (2006).

“Ash” consists of compounds that are not organic or water, generally produced by combustion of biological materials. Ash may be determined by any number of methods known by those of skill in the art, for example, as published by OMA.

The compositions of the present invention may also contain one or more minerals, micronutrients and/or trace elements, e.g. iodine, selenium, vitamin D, vitamin A, niacin, thiamine, pantothenic acid, pyridoxine, riboflavin, folic acid, biotin, vitamin B12, calcium, phosphorus, sodium, chloride, potassium, magnesium, manganese, copper, zinc or iron salts. One useful trace element is manganese. Manganese is essential to a host of enzymes as a cofactor, which may regulate the metabolism of foods, including proteins, fats, and carbohydrates. Such enzymes may include oxidoreductases, transferases, hydrolases, lyases, isomerases, ligases, lectins, and integrins. Manganese also affects bone development and neurological function. Manganese may be naturally present in the components of the compositions, or it may be added to compositions. Methods of measuring manganese content in a composition are well known to those of skill in the art, for example, as published by OMA.

The compositions of the present invention may also include vitamins and minerals in amounts required to avoid deficiency and maintain health. These amounts, and methods of measurement are known by those skilled in the art. For example, AAFCO provides recommended amounts of such ingredients for dogs and cats. As contemplated herein, useful vitamins may include, but are not limited to, vitamin A, vitamin B₁, vitamin B₂, vitamin B₆, vitamin B₁₂, vitamin C, vitamin D, vitamin E, vitamin H (biotin), vitamin K, folic acid, inositol, niacin, choline, and pantothenic acid. Dietary supplements of vitamin and mineral “premixes” which meet AAFCO recommended nutritional requirements may be used in the compositions disclosed herein and are familiar to one of skill in the art.

The compositions of the present invention may additionally comprise additives, stabilizers, fillers, thickeners, flavorants, palatability enhancers and colorants in amounts and combinations familiar to one of skill in the art.

The antioxidant enriched compositions employed in the methods of the present invention may be in the form of a food or pet food. In another embodiment, the composition is a treat. Treats are known to those skilled in the art, and can include, for example, compositions that are given to an animal to eat during non-meal time, e.g., a dog biscuit.

While compositions of any consistency or moisture content are contemplated, preferably the food compositions of the present invention may be, for example, a wet, semi-moist, or dry animal food composition. “Wet” food refers to food that has a moisture content of about 70 to 90%. “Semi-moist” food refers to food that has a moisture content of about 15% to 40%. “Dry” food refers to compositions about 5% to 15% moisture content and is often manufactured in the form of small bits or kibbles. Also contemplated herein are compositions that may comprise components of various consistency as well as components that may include more than one consistency, for example, soft, chewy meat-like particles as well as kibble having an outer cereal component and an inner cream component as described in, e.g., U.S. Pat. No. 6,517,877. The kibble may then be dried and optionally coated with one or more topical coatings known by those skilled in the art, for example, flavors, fats, oils, powders, and the like. The compositions of the present invention can be prepared using conventional manufacturing methods.

In addition to administering an antioxidant enriched composition, the method of the present invention comprises treating the animal to behavioral enrichment. As discussed above, “behavioral enrichment” includes, but is not limited to, subjecting an animal to one or more series of activities that can provide mental and/or physical stimulation of the animal that is beneficial to the well being of the animal, mentally or physically. Such activities may include exercise of any type, (e.g., running or playing with kennel mates or human companions, hand walking by leash for a period of time, e.g., 30 minutes or longer several times a week), environmental enrichment (e.g., providing the animal with new toys which engage the animal's mental faculties) as well as a programs or activities that provide cognitive enrichment. Cognitive enrichment for an animal may include, but is not limited to, e.g., oddity determination learning problems, other cognitive exercise or any behavioral enrichment as described above. Additional appropriate types of behavioral enrichment for an animal are familiar to one of skill in the art. See, generally Zicker. S C, Prog Neuropsychopharmacol Biol Psychiatry, 2005 March; 29(3):455-9.

The present invention also includes kits comprising an animal feed composition enriched in antioxidants and instructions to enhance disease resistance in the animal fed the animal feed composition by providing behavioral enrichment, including but not limited to, exercising the animal regularly to cause behavioral enrichment. The components of a kit may be physically associated in or with one or more containers and considered as a unit of manufacture, distribution, sale, or use. Containers include, for example, bags, boxes, bottles, shrink wrap packages, stapled or otherwise fixed components, and combinations thereof. A single package can be employed, for example, containers or individual food compositions physically associated such that they are considered a unit for manufacture, distribution, sale, or use. The kit may include instructions on the package or included as a package insert, or optionally, the instructions may be in the form of a web address to which the user of the animal feed composition is directed. The kit may be promoted, distributed, or sold as a unit for performing the methods of the present invention.

### EXAMPLES

### Materials and Methods

Animals. Twenty-one (12 males, 9 females) healthy, geriatric (10- to 13-year-old) beagles are maintained according to currently accepted practices of good animal husbandry. All dogs are determined to be free of chronic systemic disease based upon physical examinations, complete blood counts, serum biochemical evaluations, urinalyses and fecal examinations for parasites.

These dogs are part of a longitudinal study of canine cognition that included both a nutritional and a behavioral intervention. The present investigation starts with a period of baseline testing, which is used to separate dogs into four cognitively equivalent groups based on their performance on a series of neuropsychological tests. There are two treatment variables: dietary fortification and behavioral enrichment. Food groups include a control food and an antioxidant-fortified food as described in Table 1:

TABLE 1
Control and Antioxidant Enriched Diets

| Ingredient (% unless | | |
|---|---|---|
| otherwise indicated) | Test | Control |
| Corn | 62 | 68 |
| Poultry by-product meal | 11 | 11 |
| Rice | 5 | 5 |
| Soybean mill run | 4 | 4 |
| Corn Gluten Meal | 3 | 3 |
| Soybean Oil | 2 | 2 |
| Pork Fat | 2 | 2 |
| Egg | 1 | 1 |
| Dried Carrots | 1 | 0 |
| Dried Spinach | 1 | 0 |
| Dried Grape Pomace | 1 | 0 |
| Dried Tomato Pomace | 1 | 0 |
| Dried Citrus Pulp | 1 | 0 |
| Palatability Enhancer | 1 | 1 |
| Flaxseed | 1 | 1 |
| Vitamin/Mineral Premix | 3 | 2 |
| Vitamin E (IU/Kg) | 1000 | 250 |
| Vitamin C (ppm) | 200 | 0 |
| Taurine (added ppm) | 600 | 0 |
| L-Carnitine (ppm) | 300 | 0 |
| alpha-lipoic acid (ppm) | 130 | 0 |

Dietary intervention. The two foods are formulated to meet the adult maintenance nutrient profile for the American Association of Feed Control Officials recommendations for adult dogs. Control and test foods are identical in composition, except for the inclusion of a broad-based antioxidant and mitochondrial cofactor supplementation in the test food. The food is produced according to conventional methods by an extrusion process and a production batch is fed for no more than 6 months before a new lot is manufactured.

Behavioral enrichment modification consists of groups receiving either a normal level of behavioral/cognitive stimulation or an enhanced program of behavioral/cognitive enrichment. Thus, 4 groups of dogs are created in a 2×2 factorial design, with 5 or 6 animals per group. Group C-C dogs are fed the control food and provided with the normal level of behavioral/cognitive experience; group C-E dogs receive the control food and a program of supplemental behavioral/cognitive experience; group A-C dogs are fed the antioxidant-fortified food and are also given the normal behavioral/cognitive experience; and, group A-E dogs receive both the antioxidant-fortified food and the supplemental behavioral/cognitive experience.

Dogs are fed once daily under supervision and water is available ad libitum. Dogs are weighed at seven-day intervals and caloric intake is adjusted to maintain body weight. Each animal is observed daily for changes in general appearance and behavior. Individual records are maintained for each animal and the amount of food offered to each dog is recorded.

Behavioral intervention. Behavioral enrichment commences after completion of the baseline cognitive testing, at the start of the treatment phase. The animals allocated to the enriched treatment groups are housed with kennel mates, exercised by hand walking with a leash for at least 30 minutes twice a week, and given sets of toys in their home room, which are rotated every week. Cognitive enrichment also includes an enrichment protocol, which starts following the baseline testing with a series of landmark discrimination problems, according to conventional methods (Milgram et al., Neurosci. Biobehav. Rev., 26:679-695 (2002)) for 6 of 7 days each week. After completing these tasks, the animals are trained on a series of oddity discrimination learning problems, as previously described (Milgram et al., Neurobiol. Aging, 23:737-745 (2002). Not all of these behavioral enrichments are provided at the same frequency to animals in the normal behavioral enrichment groups. The dogs in the control groups receive approximately 3 to 4 months of cognitive testing each year to complete the annual assessments.

### Immunological Measurements

Isolation of peripheral blood mononuclear cells (PBMCs). PBMCs are isolated using a modification of the methods of J. E. Coligan et al., Current Protocols in Immunology, New York: John Wiley and Sons, 1992; and S. Krakowka et al., Vet. Immunol. Immunopathol., 15:181-201 (1987). Briefly, 3 mL of Histopaque® 1077 (Sigma Chemical Co., St. Louis, Mo., U.S.A.) are added to 15 mL centrifuge tubes and under layered with 3 mL of Histopaque® 1119 (Sigma Chemical Co., St. Louis, Mo., U.S.A.). Cells are separated from whole blood collected in EDTA by layering 6 mL of undiluted blood over the Histopaque® double gradient media. Tubes are centrifuged at 700×g for 30 min at 20° C. Cells harvested from the mononuclear layer are washed in phosphate buffered saline (PBS) solution (pH 7.4), and then centrifuged at 400×g for 10 min at 20° C. The resultant cell pellets are resuspended in 1 mL of RPMI-10 (RPMI-1640 media with L-glutamine and 10% fetal bovine serum, Hyclone, Logan, Utah, U.S.A.), containing 10 mM HEPES buffer (pH 7.4) and 1% penicillin-streptomycin. A 40 μL aliquot of the cell suspension is used to determine cell concentration (Coulter ZB1 Counter, Coulter Electronics, Inc, Hialeah, Fla., U.S.A.). Another 20 μL aliquot is used to assess purity of PBMCs (differential cell count) by microscopic examination after Wright-Giemsa staining and cell viability after trypan blue exclusion.

Lymphocyte stimulation and measurement of proliferation and apoptosis by flow cytometry. Triplicate wells of PBMCs are prepared in 96-well tissue culture plates at a concentration of 5×10⁶ PBMC/mL. Cells (1×10⁶) are either stimulated with 2.5 μg/mL of Con A (Pharmacia Biotech, Alameda, Calif. U.S.A.) in RPMI-10 or treated with 1.0×10⁻⁶ M dexamethasone (Sigma Chemical Co., St. Louis, Mo., U.S.A.) in RPMI-10, or cells are treated with both 2.5 μg/mL Con A and 1.0×10⁻⁶ M dexamethasone in RPMI-10. Cells treated with RPMI-10 alone served as controls. Final volume in each well is 250 μL. Plates are incubated for 48 h at 37° C. in an atmosphere of 5% CO₂. After incubation, microplates are centrifuged at 200×g for 3 min at 20° C. Cells are washed twice with 200 μL PBS and then resuspended in 100 μL PBS and 100 μL Cytochex™ (a white blood cell preservative. Streck Laboratories. Inc., La Vista, Nebr., U.S.A.). Microplates are sealed and stored at 2° to 8° C. for subsequent flow cytometry analysis. Before flow cytometry, plates are centrifuged at 200×g for 3 min at 20° C. Supernatants are discarded and 200 μL of 7.5 μM propidium iodide (PI) staining solution containing 5 mM EDTA, 50 μg/mL RNase A and 0.3% w/v saponin are added to each well. Plates are then incubated in the dark for 30 minutes at 20° C. After incubation, plates are analyzed by flow cytometry using a FACSCalibur™ flow cytometer (Becton-Dickinson, San Jose, Calif. U.S.A.). Data are analyzed using CellQuest™ software (Becton-Dickinson, San Jose, Calif.). Three cell populations, apoptotic, resting, and proliferating, are identified by the amount of PI taken up by the DNA.

Lymphocyte stimulation and analysis of CD4, CD8, B cell, and CD69 surface marker expression by flow cytometry. Duplicate wells of 5×10⁵ PBMCs each are prepared in 96-well microplates. Cells are stimulated by adding 2.5 μg/mL Con A (final concentration) in a total volume of 200 μL/well. Microplates are incubated for 48 h at 37° C. in 5% CO₂. Duplicate wells of 5×10⁵ PBMC are also prepared for assessing surface marker expression at 0 hours. No mitogen is added to these wells. These non-stimulated cells, which represented baseline expression of cell surface markers, are stained in the same manner as stimulated cells.

Cells are stained with either 25 μL of fluorescein-labeled monoclonal antibody against canine CD4, CD8, or B-cells, and biotin-labeled monoclonal antibody against CD69 (Anti-canine CD4-FITC(CM 12.125); Anti-canine CD8-FITC(CM 1.140); Anti-canine mature B-cell marker-FITC(CM 11.425); and Anti-cat/dog B/T activation marker CD69-biotin labeled (CM 2.58), (Custom Monoclonals International, West Sacramento, Calif., U.S.A.), and incubated on ice for 20 min. Microplates are then centrifuged at 200×g for 3 min at 20° C. Supernatants are discarded and microplates vortexed. Cells are washed twice with 200 μL PAB solution (PBS containing 0.1% sodium azide and 1.0% bovine serum albumin) followed each time by centrifugation at 200×g for 3 minutes at 20° C. Cells in each well are then resuspended in 100 μL PAB. Streptavidin-spectral red (Streptavidin-APC; FITC-labeled Mouse IgG1, (kappa) isotype control; and biotin-labeled mouse IgG1, (kappa) isotype control, PharMingen, San Diego, Calif., U.S.A.) (4 mM) is added as the second-step reagent for biotin-labeled monoclonal antibodies (50 μL/well), and microplates are incubated on ice for 20 min. Following incubation, microplates are washed twice with PAB. Following the last wash, supernatants are aspirated, plates are vortexed and 100 μL PAB and 100 μL Cytochex™ are added to each well. Microplates are sealed and stored at 2 to 8° C. until subsequent processing and flow cytometric analysis can be completed. Additional wells are stained with isotype-matched control antibodies to determine background fluorescence. The percentage of FITC-positive CD4, CD8, and B cells are determined by selecting cells that fall outside the range of background isotype fluorescence. These cells are then analyzed for CD69 expression. Again, isotype control fluorescence is used as a boundary. The level of CD69 expression, or mean cell fluorescence, is also measured. Data are analyzed using CellQuest™ software.

Quantitative measurement of tumor necrosis factor (TNF)-α production by stimulated mononuclear cells. PBMCs are aliquoted into 6-well tissue culture plates (1×10⁶ cells/well). For stimulation of TNF-αproduction, LPS from Escherichia coli 055:B5 in RPMI-10 media (final concentration 30 μg/mL) is added to 3 wells of each plate, for a final volume of 2 mL/well. Control wells are prepared without adding LPS to cells. Plates are incubated at 37° C. for 24 h in 5% CO₂. Previous timed incubation studies in the laboratory showed maximal production of TNF-α at 24 h. After incubation, plates are centrifuged at 200×g for 10 min at 20° C. Aliquots of supernatant from each well are stored at −70° C. for subsequent TNF-α analysis.

TNF-αproduction by PBMCs is determined by following the procedures recommended by the manufacturer (Human TNF-αELISA kit. Pierce Endogen, Rockford, Ill., U.S.A.) of the TNF-αkit. Optical density is read at wavelengths 450 and 550 nm. Concentrations for TNF-α are determined using the SoftMax®-Pro software program (SPECTRAmax 340 PC microplate reader, Molecular Devices Corporation, Sunnyvale, Calif., U.S.A.). Results from two control wells and two LPS-stimulated wells are reported.

Isolation of peripheral blood neutrophils for assessment of phagocytosis. Three mL of Histopaque® 1077 (Sigma Chemical Co., St. Louis, Mo.) are added to 15 mL centrifuge tubes and under layered with 3 mL of Histopaque® 1119 (Sigma Chemical Co., St. Louis, Mo.). Neutrophils are separated from whole blood by layering 6 mL of undiluted whole blood in EDTA over the double gradient media. Tubes are centrifuged at 700×g for 30 min at 20° C. After removing the PBMC layer, neutrophils are collected and washed with a buffered 0.83% ammonium chloride solution (pH 7.4) until contaminating RBCs are lysed and cleared. Cells are then washed twice with PBS, centrifuged at 400×g for 10 min at 20° C. and resuspended in a final volume of 1 mL of RPMI-10. Measurements for neutrophil concentration, purity, and viability are preformed as previously described for PBMCs.

Neutrophil phagocytosis of opsonized latex beads determined by flow cytometry. Neutrophils (2×10⁵ in 100) are dispensed into triplicate wells of 96-well tissue culture plates. Yellow-orange latex Fluoresbrite® beads are diluted 1:1000 in PBS to a concentration of 6.59×10⁹ beads/mL PBS, and then opsonized by adding an equal volume of 50% laboratory-control canine serum at 37° C. for 30 min. The beads are then diluted 1:4 in Hanks balanced salt solution (HBSS) with 0.14 g/L CaCl₂ (pH 7.4). The resulting bead solution (100 μL) is added to wells containing cells for a final bead to cell ratio of 25:1. Plates are incubated at 37° C. for 30 min. and then cells are washed twice in 200 μL cold PBS, centrifuged at 200×g for 3 min, and resuspended in 100 μL PAB. Primary antibody (Canine Neutrophil Stain, Cell Line CADO48A, VMRD, Inc., Pullman, Wash., U.S.A.) (25 μL) is added to appropriate wells and plates are incubated on ice for 20 min. Plates are centrifuged at 200×g for 3 min. supernatants discarded, and then vortexed. PAB (200 μL) is added to all wells and plates are centrifuged at 200×g for 3 min. After washing twice, cells are resuspended in 100 μL PAB. Next 50 μL of secondary antibody, goat F(ab′)2 anti-mouse IgG-APC (Caltag Labs, Burlingame, Calif., U.S.A.), are added to all wells, except those containing beads only. Plates are incubated on ice for 20 min and then washed as previously described. After the final wash step, 100 μL PAB and 100 μL Cytochex™ are added to each well, and plates are sealed and stored at 2° to 8° C. for subsequent flow cytometric analysis. Data are analyzed using CellQuest™ software.

Quantitative measurement of LTB₄ production by stimulated neutrophils. Neutrophils are isolated as described above, except that after RBCs are lysed and cleared, cells are washed twice in HBSS without CaCl₂, centrifuged at 400×g for 10 min at 20° C. and resuspended in a final volume of 1 mL of HBSS with 0.8 mM CaCl₂. Measurements for cell concentration, cell purity, and cell viability are performed as previously described. Aliquots of 5×10⁶ neutrophils are transferred to 5 mL polypropylene tubes and the volume adjusted to 495 μL with HBSS containing 0.8 mM CaCl₂. Neutrophils are then stimulated with 5 μL of calcium ionophore A23187 (Sigma Chemical Co., St. Louis, Mo.) in 0.2% dimethyl sulfoxide such that the final concentration of A23187 is 10 μM. Unstimulated neutrophils received 5 μL of 0.2% dimethyl sulfoxide without calcium ionophore. All rubes are incubated for 5 min in a 37° C. water bath and the reaction is terminated by addition of 2 mL of ice-cold methanol to each tube followed by incubation on ice for 20 min. Tubes are centrifuged for 5 min at 1000×g and the supernatants are transferred to 5-mL polypropylene tubes and stored at −70° C. until subsequent LTB₄ measurements are made.

Leukotriene B₄ is extracted, separated, and quantified (Jha et al., Prostaglandins Leukot. Essent. Fatty Acids (2005)). A standard calibration curve for LTB₄ is made by adding 100 ng of prostaglandin (PG) B₃ (Cayman Chemical Co., Ann Arbor, Mich., U.S.A.) as an internal standard to samples containing 6.25 to 100 ng of LTB₄ (Sigma Chemical Co., St. Louis, Mo.). Prostaglandin B₃ is chosen as the internal standard because it is widely separated from LTB₄ present in actual samples during high performance liquid chromatography (HPLC) separation (T. Terano et al., Biochem. Pharmacol, 33:3071-3076 (1984)). The standard solutions are extracted as above and LTB₄ is detected by HPLC. The peak area ratio for LTB₄/PGB₃ is calculated and plotted against the concentration of LTB₄. The concentration of LTB₄ in test samples is calculated with reference to the standard curve. Final LTB₄ concentration in samples is reported as nanograms of LTB₄ per 5×10⁶ cells.

Quantitative measurement of plasma C-reactive protein (CRP). Concentrations of CRP are determined from plasma aliquots that are frozen soon after collection and stored at −70° C. until subsequent analysis, following the procedures recommended by the manufacturer of the canine CRP assay kit (Canine C-Reactive Protein Immunoassay Kit, Tri-Delta Diagnostics. Inc., Morris Plains, N.J., U.S.A.). All plasma samples are diluted 1:100 based on values previously obtained in the laboratory. Optical density is read at wavelengths 450 and 630 nm. Concentrations of CRP are determined using the SoftMax®-Pro software program (Molecular Devices Corporation, Sunnyvale, Calif., U.S.A.).

Statistical Analysis. Data are reported as means±SEM. Using the Kolmogorov-Smirnov Test, data that are found to be normally distributed are analyzed using analysis of variance. On the basis of the Modified Levene Equal-Variance test, data that are homoscedastic are analyzed using Analysis of Variance followed by post hoc separation of the means using the Tukey-Kramer Multiple-Comparison test. When the assumptions of normality and equal variance are suspect, data are analyzed by the nonparametric Kruskal-Wallis Test, followed by post hoc separation of the means using the Kruskal-Wallis Z Test. Overall significance is set at P<0.05. Statistical analyses are performed using the Number Cruncher Statistical System (NCSS), version 2004.

### Results

Lymphocyte stimulation and measurement of proliferation and apoptosis by flow cytometry. The percentages of apoptotic lymphocytes and proliferating lymphocytes after stimulation with Con A, or suppression with Dex, or both stimulation with Con A and suppression with Dex, after 48 hours incubation are shown in Table 2 below. Simultaneous stimulation with Con A and suppression with Dex results in decreased proliferation of lymphocytes from dogs in group A-E that received both the antioxidant-enriched food and cognitive enrichment, compared to dogs in groups A-C and C-E (dogs receiving the antioxidant-enriched food, and dogs receiving cognitive enrichment only) (P=0.03).

TABLE 2
Percentage of lymphocytes that are apoptotic and proliferating
after 48 hours incubation with concanavalin A (Con A), or
dexamethasone (Dex), or both (Con A/Dex), as determined by
propidium iodide staining and flow cytometry.

| | | | | |
|---|---|---|---|---|
| | | | Con A/Dex | |
| | Con A | Dex | (2.5 μg/mL/ | |
| Group* | (2.5 μg/mL) | (1.0 × 10⁻⁶ M) | 1.0 × 10⁻⁶ M) | Media Control |

% Lymphocytes Apoptotic (Mean ± SEM)

| | | | | |
|---|---|---|---|---|
| C-C | 4.48 ± 2.05 | 11.04 ± 2.28 | 9.23 ± 1.96 | 11.24 ± 2.50 |
| A-C | 8.74 ± 2.05 | 11.05 ± 2.28 | 10.34 ± 1.96 | 10.53 ± 2.50 |
| C-E | 8.34 ± 2.51 | 7.04 ± 2.79 | 5.43 ± 2.40 | 6.57 ± 3.05 |
| A-E | 5.40 ± 2.25 | 7.40 ± 2.50 | 5.65 ± 2.14 | 7.34 ± 2.73 |

% Lymphocytes Proliferating (Mean ± SEM)

| | | | | |
|---|---|---|---|---|
| C-C | 1.43 ± 0.42 | 1.26 ± 0.33 | 1.56 ± 0.34^{ab} | 2.85 ± 0.59 |
| A-C | 2.22 ± 0.42 | 1.52 ± 0.33 | 2.35 ± 0.34^{a} | 1.63 ± 0.59 |
| C-E | 1.48 ± 0.51 | 0.81 ± 0.40 | 2.22 ± 0.42^{a} | 0.93 ± 0.72 |
| A-E | 2.28 ± 0.46 | 1.25 ± 0.36 | 0.97 ± 0.38^{b} | 1.43 ± 0.65 |

*Group C-C = control dogs; Group A-C = dogs receiving antioxidant-enriched food; Group C-E = dogs receiving cognitive enrichment only; and Group A-E = dogs receiving both antioxidant-enriched food and cognitive enrichment.
^{a,b}Values are significantly different at P < 0.05.

Lymphocytes from dogs in group C-C (control dogs), exhibit a significantly higher percentage of apoptotic cells at 48 hours with media only treatment, with Dex treatment, and with simultaneous Con A and Dex treatment, compared to treatment with Con A alone (P=0.03). There are no significant differences in percent apoptosis at 48 hours within the other three groups of dogs after the various cell treatments. Lymphocytes from dogs in group C-E (dogs receiving cognitive enrichment only) showed a significantly higher proliferation percentage at 48 hours following simultaneous treatment with Con A and Dex compared to Dex or media alone treatment (P=0.03). There are no significant differences in percent proliferation at 48 hours within the other three groups of dogs after the various cell treatments.

Lymphocyte stimulation and analysis of CD4, CD8, B cell, and CD69 surface marker expression by flow cytometry. The effect of lymphocyte stimulation with Con A on the percentages of cells positive for surface marker expression (B cell+, CD4+, CD8+) at baseline and after 48 hours incubation is shown in Table 3 below. Cells that are B cell+, CD4+, or CD8+ are then analyzed for the presence of CD69, as well as the level of CD69 expression, i.e., mean fluorescent intensity (MFI). The difference in levels of marker expression between 48 and 0 hour incubation times are also compared.

TABLE 3
Percentage of lymphocytes expressing surface markers (B cell+, CD4+, CD8+)
at baseline and after 48 hours incubation with concanavalin A (Con A) (Mean ± SEM). as
determined by flow cytometry. Positive cells are then analyzed for the presence of
CD69. as well as the level of CD69 expression, i.e., mean fluorescent intensity (MFI).

| | | | | | | | | | |
|---|---|---|---|---|---|---|---|---|---|
| | | % B | B cell+ | | % | CD4+ | | % | CD8+ |
| | % B | cell+ | CD69+ | % | CD4+ | CD69+ | % | CD8+ | CD69+ |
| Group* | cell+ | CD69+ | (MFI) | CD4+ | CD69+ | (MFI) | CD8+ | CD69+ | (MFI) |

0 Hours Incubation (Baseline)

| | | | | | | | | | |
|---|---|---|---|---|---|---|---|---|---|
| C-C | 4.55 ± 1.36^{ab} | 0.35 ± 0.18 | 1.34 ± 1.28 | 26.88 ± 6.24 | 0.32 ± 0.13 | 2.46 ± 1.08 | 25.35 ± 5.13 | 0.34 ± 0.20 | 4.11 ± 3.72 |
| A-C | 4.10 ± 1.36^{ab} | 0.62 ± 0.18 | 4.66 ± 1.28 | 37.68 ± 6.24 | 0.30 ± 0.13 | 3.65 ± 1.08 | 28.02 ± 5.13^{#} | 0.40 ± 0.20 | 4.44 ± 3.72 |
| C-E | 1.93 ± 1.67^{a} | 0.21 ± 0.22 | 0.24 ± 1.57 | 23.39 ± 7.64 | 0.12 ± 0.16 | 2.22 ± 1.32 | 11.67 ± 6.28 | 0.55 ± 0.25 | 1.56 ± 4.55 |
| A-E | 7.84 ± 1.49^{b} | 0.23 ± 0.20 | 1.83 ± 1.40 | 23.64 ± 6.83 | 0.31 ± 0.15 | 3.47 ± 1.18 | 12.70 ± 5.62^{#} | 0.57 ± 0.22 | 14.49 ± 4.07 |

48 Hours Incubation

| | | | | | | | | | |
|---|---|---|---|---|---|---|---|---|---|
| C-C | 6.08 ± 1.05^{a} | 1.40 ± 0.55 | 2.46 ± 1.06 | 21.05 ± 6.72 | 0.65 ± 0.27 | 1.89 ± 1.02 | 25.27 ± 3.94 | 0.34 ± 0.15 | 12.39 ± 4.53 |
| A-C | 4.50 ± 1.05^{a} | 1.10 ± 0.55 | 1.07 ± 1.06 | 42.27 ± 6.72 | 0.18 ± 0.27 | 1.27 ± 1.02 | 20.10 ± 3.94^{#} | 0.27 ± 0.15 | 5.71 ± 4.53 |
| C-E | 1.19 ± 1.29^{b} | 1.41 ± 0.67 | 4.24 ± 1.30 | 33.69 ± 8.24 | 0.45 ± 0.33 | 2.86 ± 1.24 | 12.31 ± 4.83 | 0.37 ± 0.18 | 2.63 ± 5.55 |
| A-E | 6.30 ± 1.15^{a} | 0.81 ± 0.60 | 1.61 ± 1.16 | 31.35 ± 8.37 | 0.24 ± 0.29 | 2.69 ± 1.11 | 19.78 ± 4.32^{#} | 0.47 ± 0.16 | 9.47 ± 4.97 |

*Group C-C = control dogs: Group A-C = dogs receiving antioxidant-enriched food: Group C-E = dogs receiving cognitive enrichment only; and Group A-E = dogs receiving both antioxidant-enriched food and cognitive enrichment.
^{a,b}Values are significantly different at P < 0.05;
^{#}The percentage of CD8 cells after 48 hour incubation with Con A compared to 0 hour is increased in dogs receiving both dietary and behavior enrichment (group A-E) compared to dogs receiving dietary enrichment alone (group A-C: decreased).

At 0 hours (baseline), the percentage of B cells+ is significantly higher in A-E dogs receiving both the antioxidant-enriched food and cognitive enrichment compared to C-E dogs receiving cognitive enrichment alone (P<0.05). At 48 hours, the percentage of B cells+ is also significantly higher for C-C dogs (control) and for A-C dogs fed the antioxidant-enriched food (in addition to A-E dogs receiving both antioxidant-enriched food and cognitive enrichment), compared to C-E dogs receiving cognitive enrichment alone (P=0.02). Compared to time 0 values, the B cell+ CD69 MFI at 48 hours change significantly for A-C dogs receiving the antioxidant-enriched food (decreased) compared to C-E dogs receiving cognitive enrichment alone (increased) (P=0.049).

There are no significant differences among the four groups of dogs for CD4+ cells. For CD8+ cells, the only significant finding is that compared to time 0 values, the % CD8+ cells at 48 hours change for A-C dogs receiving the antioxidant-enriched food (decreased) compared to A-E dogs receiving both the antioxidant-enriched food and cognitive enrichment (increased) (P=0.05).

Quantitative measurement of TNF-α production by stimulated mononuclear cells. No significant differences are detected among the four groups of dogs for production of TNF-α by stimulated PBMCs. TNF-α concentration is 289±57 pg/mL (mean±SEM) for C-C dogs (control), 230±57 for AC dogs receiving the antioxidant-enriched food, 412±69 for C-E dogs receiving cognitive enrichment only, and 374±62 for A-E dogs receiving both the antioxidant-enriched food and cognitive enrichment.

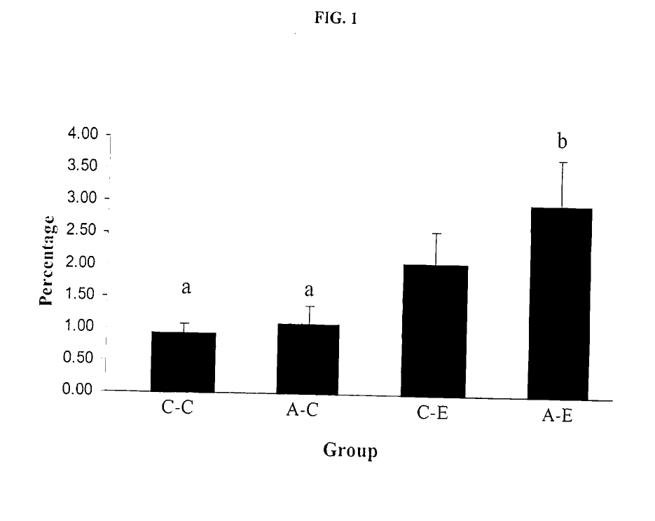

Neutrophil phagocytosis of opsonized latex beads determined by flow cytometry. The percentage of neutrophils that phagocytize Fluoresbrite® beads is significantly different among the four groups of dogs (P=0.02) (FIG. 1). A-E dogs that receive both the antioxidant-enriched food and cognitive enrichment show increased phagocytosis compared to C-C dogs (control) and A-C dogs receiving the antioxidant-enriched food, but no cognitive enrichment.

Mean fluorescent intensity values are not significantly different among the four groups of dogs. Mean fluorescent intensity is 168±24 (mean±SEM) for C-C dogs (control), 152±24 for A-C dogs receiving the antioxidant-enriched food, 140±29 for C-E dogs receiving cognitive enrichment only, and 103±26 for A-E dogs receiving both the antioxidant-enriched food and cognitive enrichment.

Quantitative measurement of LTB₄ production by stimulated neutrophils. Production of LTB₄ by stimulated peripheral blood neutrophils is measured and there are no differences among the four groups of dogs. LTB₄ concentration is 12.3±1.9 ng per 5×10⁶ neutrophils (mean±SEM) for C-C dogs (control), 18.8±1.9 for A-C dogs receiving the antioxidant-enriched food, 14.8±2.4 for C-E dogs receiving cognitive enrichment only, and 14.3±2.1 for A-E dogs receiving both the antioxidant-enriched food and cognitive enrichment.

Quantitative measurement of plasma CRP. Plasma CRP concentration (mean±SEM) for C-C dogs (control) is 2.14±1.20 μg/mL, for A-C dogs receiving antioxidant-enriched food 4.51±1.20 μg/mL, for C-E dogs receiving cognitive enrichment only 2.29±1.47 μg/mL, and for A-E dogs receiving both cognitive enrichment and antioxidant-enriched food 1.83±1.32 μg/mL. There are no significant differences among the four groups of dogs.

Thus, dogs are enrolled in a 2 year longitudinal study that include both a nutritional (control food or antioxidant-fortified food) and a behavioral (normal level or cognitive enrichment) intervention. Behavior enrichment included increased exercise, environmental enrichment, and a series of learning tasks. Immunological measurements are performed on peripheral blood cells.

Neutrophil phagocytosis of opsonized latex-coated beads is significantly increased in dogs receiving both dietary antioxidants and cognitive enrichment. The combination of dietary antioxidants and cognitive enrichment is more effective than either intervention alone, and results in improved neutrophil cell function, cells involved in the first line of defense against pathogens. Lymphocytes are stimulated with concanavalin A (Con A) and their resistance or susceptibility to dexamethasone (Dex)-induced cell death (apoptosis) is determined. Simultaneous stimulation of cells with Con A and suppression with Dex results in decreased lymphocyte proliferation in dogs receiving both dietary antioxidants and cognitive enrichment, compared to dogs receiving dietary antioxidants or cognitive enrichment alone. There are no significant differences between the groups for percentages of CD4+ and CD8+ T-lymphocyte subpopulations before or after lymphocyte stimulation with Con A. However, prior to lymphocyte stimulation with Con A, the percentage of B cells is higher in dogs receiving both dietary antioxidants and cognitive enrichment compared to dogs receiving cognitive enrichment alone, and this change persists after stimulation with Con A for 48 h. Production of tumor necrosis factor (TNF)-α by lipopolysaccharide (LPS)-stimulated peripheral blood mononuclear cells, production of leukotriene (LT) B4 by calcium-ionophore-stimulated peripheral blood neutrophils, and levels of plasma C-reactive protein (CRP) are not significantly different among groups of dogs.

We have investigated the potential for diets rich in antioxidants, plus or minus cognitive enrichment, to influence inflammatory and immune responses in dogs. (J. A. Hall et al., Vet. Immunol. Immunopathol., 113(1-2):224-233 (2006)). As explained in detail in the Examples provided herein, in one set of experiments, peripheral blood lymphocytes are stimulated ex vivo with the mitogen concanavalin A (“Con A”) and their resistance or susceptibility to steroid-induced cell death (apoptosis) is determined. In a second set of experiments, lymphocytes are stimulated with Con A and cell phenotypic markers (CD4, CD8, and B cells), as well as a cell activation marker (CD69), are assessed by flow cytometry.

To determine the potential for dietary antioxidants to alter immune cell cytokine production in response to lipopolysaccharide (“LPS”) challenge, LPS-stimulated peripheral blood mononuclear cells (“PBMC”) are used to assess production of the proinflammatory cytokine tumor necrosis factor (TNF)-α. To determine if cell function is altered, i.e., the ability to ingest foreign particles, phagocytosis of opsonized latex-coated beads by peripheral blood neutrophils is measured by flow cytometry. Stimulated peripheral blood neutrophils are also used to assess leukotriene (LT) B₄ production. Finally, the concentration of C-reactive protein in plasma is measured as an indicator of inflammation.

Several tests of the immune response, including the delayed hypersensitivity skin test, antibody production, lymphocyte proliferation, cytokine production, and numbers of specific subgroups of white blood cells, e.g., subgroups of lymphocytes, are influenced by essential nutrient intake and may serve as functional tests for evaluating nutrient status (D. S. Kelley et al., Am. J. Clin. Nutr., 63(6):994S-996S (1996)). The intake of certain nutrients can be modulated to regulate the activity of the immune system. For example, to maintain their immune responses at an optimum, healthy elderly persons may need increased amounts of certain essential micronutrients (vitamin E, vitamin C, and β-carotene) that are higher than their usual dietary intake and the current recommended dietary allowance (RDA), and higher than amounts needed by younger adults.

The dietary intervention in this study consists of providing a dry maintenance dog food fortified with a broad spectrum of antioxidants and mitochondrial cofactors (S. C. Zicker, Prog. Neuropsychopharmacol. Biol. Psychiatry, 29(3):455-459 (2005); N. W. Milgram et al., Neurobiol. Aging, 26(1): 77-90 (2005)). In the examples provided herein, the two foods tested are similar, except one is antioxidant-enriched. The antioxidant-enriched food contains approximately 10-fold more d,1-α-tocopheryl acetate and 1-carnitine, 6-fold more d,1-α-lipoic acid, and 3-fold more ascorbic acid all on as as-fed basis, in addition to 1% inclusions of each of the following: spinach flakes, tomato pomace, grape pomace, carrot granules and citrus pulp compared to the control food.

The rationale for these inclusions is as follows: vitamin E is lipid soluble and acts to protect cell membranes from oxidative damage; 1-carnitine is a precursor to acetyl-1-carnitine and is involved in mitochondrial lipid metabolism and maintaining efficient mitochondrial function; α-lipoic acid is a cofactor for the mitochondrial respiratory chain enzymes, pyruvate and α-ketoglutarate dehydrogenases, as well as an antioxidant capable of redox recycling other antioxidants and raising intracellular glutathione levels; vitamin C is essential in maintaining oxidative protection for the soluble phase of cells, as well as preventing vitamin E from propagating free radical production; fruits and vegetables are rich in flavonoids and carotenoids and other antioxidants. Without being bound by theory, the effectiveness of the antioxidant-enriched food is theoretically linked to its ability to arrest or reverse cellular dysfunction produced by excessive free radicals and improvement of aged mitochondrial function.

The rationale for including cognitive and behavioral enrichment intervention in the studies provided herein is to test the hypothesis that a link exists between cognitive experience and the development of age-dependent cognitive dysfunction. It has been shown that aged dogs receiving a combined treatment of antioxidant-fortified food and behavioral enrichment displayed more accurate learning than the other groups of aged dogs. Discrimination learning is significantly improved by behavioral enrichment, whereas reversal learning is improved by both behavioral and dietary antioxidant enrichment. To our knowledge, there are no similar studies in dogs to assess the effects of behavioral enrichment on the immune response; one study demonstrated that in enriched-housed pigs, baseline salivary Cortisol concentrations differed from barren-housed pigs, but immune function appeared to be relatively unaffected (J. de Groot et al. Physiol. Behav., 71:217-223 (2000)). Thus, it was not known whether an increased dietary intake of antioxidants and mitochondrial cofactors in conjunction with behavioral enrichment in aged dogs would alter tests of their immune response.

As discussed in detail herein, ex vivo tests used to evaluate immune responses include lymphocyte proliferation and apoptosis in response to mitogen (Con A) or suppressor (Dex), changes in percentages of lymphocyte subgroups based on cell phenotypic markers (CD4, CD8, and B cells) and a cell activation marker (CD69). TNF-αcytokine production, neutrophil phagocytosis, LTB₄ production, and plasma CRP levels. Data indicate that neutrophils from dogs receiving maintenance food fortified with a broad spectrum of antioxidants and mitochondrial cofactors in combination with behavioral enrichment show increased phagocytosis compared to control dogs or dogs receiving antioxidant-enriched food alone.

Furthermore, we demonstrate herein that neutrophils from dogs fed antioxidant-enriched food in combination with behavioral enrichment have a higher percent phagocytosis than neutrophils from dogs fed antioxidant-enriched food alone.

Data also indicate that simultaneous stimulation of lymphocytes with Con A and suppression with Dex results in decreased proliferation of lymphocytes in dogs receiving both dietary and behavioral enrichment compared to lymphocytes from dogs receiving either intervention alone. This would indicate that following Con A stimulated proliferation, susceptibility to steroid-induced cell death (apoptosis) is increased in dogs receiving these two interventions.

It has been reported that chronic stress in dogs subjected to social and spatial restriction results in increased Con A-induced lymphocyte proliferation (B. Beerda et al. Physiol. Behav., 66(2):243-254 (1999)). Con A is a T cell mitogen, and enhanced mononuclear cell proliferation is assumed to result from proliferation of the T cell population. T cells are responsible for cell-mediated immunity, undergoing proliferation when stimulated, and they produce lymphokines that regulate the functions of other immune cells. In vitro, β-carotene and vitamin A enhance Con A-induced proliferation of lymphocytes from dairy cattle (J. J. Michal et al., J. Dairy Sci., 77:1408-1421 (1994): L. R. Daniel et al., J. Dairy Sci., 74:911-915 (1991)). These studies indicate that B-carotene can enhance host defense mechanisms by potentiating lymphocyte and phagocyte functions. Dietary supplementation with B-carotene also increases T-cell proliferation in rats (A. Bendich et al., J. Nutr., 116:2254-2262 (1986)) and in weanling pigs (C. D. Hoskinson et al., Fed. Am. Soc. Exp. Biol. J., 3:A663 (1989)). Lymphocyte proliferation has been shown to decrease with age in both mice and humans and increase in response to a diet supplemented with antioxidants (M. De la Fuente, Eur. J. Clin. Nutr., 56:S5-S8 (2002)).

Analysis of specific lymphocyte subgroups in the study reported herein reveal that the percentage of B cells is significantly higher before and after incubation with Con A for 48 hours in dogs receiving both dietary and behavioral enrichment compared to dogs receiving behavioral enrichment alone (0 hours) or all other groups of dogs (48 hours). The percentage of CD8+ cells after 48 hours incubation with Con A compared to 0 hours is increased in dogs receiving both dietary and behavioral enrichment compared to dogs receiving dietary enrichment alone (decreased). We have previously shown that dogs consuming moderate (101 mg/kg of food) or high (447 mg/kg of food) concentrations of α-tocopheryl acetate had increased percentages of CD8+ cells compared with dogs consuming low-normal (17 mg/kg of food) α-tocopheryl acetate concentrations (J. A. Hall et al., Am. J. Vet. Res., 64(6):762-772 (2003)). From that study we concluded that an optimum amount of dietary α-tocopheryl acetate concentration stimulates the CD8+ T cell population. In the current study, the antioxidant-enriched food contains twice as much vitamin E (1000 ppm) as in the previous study. The mechanism(s) by which vitamin E enhances the immune response have yet to be fully explained; however, evidence suggests that it works by reducing prostaglandin synthesis, decreasing the formation of free radicals, or both (S. N. Meydani et al., Am J Clin Nutr 1990; 52:557-63: M. Meydani, Mech. Ageing Dev., 111:123-32 (1999)). Dietary supplementation with vitamin E has been shown to improve T cell responsiveness by reducing macrophage PGE₂ production (A. Beharka et al., Methods Enzymol., 282:247-63 (1997)). Reactive oxygen species, especially hydrogen peroxide produced by activated macrophages, also depressed lymphocyte proliferation (Z. Metzger et al., J. Immunol., 124:983-8 (1998)).

Production of LTB₄ from neutrophils of dogs fed the two diets is not altered, most likely because there were similar amounts of precursor fatty acids in both diets. Production of LTB₄ from canine peripheral blood neutrophils reflects the plasma concentration of substrates, i.e, arachidonic acid, from which they are derived. In turn, the plasma content of arachidonic acid is dependent on the dietary concentrations of these fatty acids. The concentrations of LTB₄ produced by stimulated neutrophils of dogs from this study are similar to those previously reported for dogs.

The prototype marker of inflammation is C-reactive protein. In addition, inflammation is associated with increased pro-inflammatory cytokine release (IL-1, IL-6, and TNF-α). Vitamin E therapy in humans with diabetic vasculopathies results in a reduction of pro-inflammatory cytokine and CRP levels (I. Jialal et al., Free Radic. Res., 36:1331-1336 (2002)). Healthy adults supplemented with vitamins C and E show increased production of TNF-α by their peripheral blood mononuclear cells (G. J. Kee-Ching et al., Am. J. Clin. Nutr., 64:960-965 (1996)). The lack of effect of antioxidant or behavioral interventions on markers of inflammation (TNF-α and CRP) evaluated in this study does not suggest that these interventions are not beneficial for protection against senescent deterioration of the immune system. An immunomodulatory role of antioxidants has been proposed because antioxidants are able to raise the decreased functions and lower the very stimulated functions in immune cells from aged animals. If the levels are within the normal range, an immunomodulatory role would be difficult to demonstrate. The plasma concentrations of CRP for beagles in this study are within the normal range reported (S. Yamamoto et al., Vet. Res. Commun., 17:85-93 (1993)) for the dog (2.4 to 30.0 μg/ml).

As described herein, the behavioral enrichment intervention includes a program of cognitive enrichment, increased physical activity and environmental enrichment. The food is enriched with a cocktail containing both antioxidants and mitochondrial cofactors. In summary, dietary antioxidant enrichment with mitochondrial cofactors in conjunction with behavioral enrichment increases neutrophil phagocytosis and B cell percentages in animals. The percentage of CD8+ cells increases after 48 hours incubation with Con A compared to 0 hours in dogs receiving both antioxidant and behavioral enrichment, whereas the percentage decreases in dogs receiving antioxidant enrichment alone. These results show that dietary and behavioral enrichment enhance host defense mechanisms.

While particular embodiments of the present invention have been shown and described herein, it will be apparent to those skilled in the art that changes and modifications may be made without departing from the broader aspects of invention.

1. A method to enhance disease resistance in an animal comprising
a) feeding said animal a diet enriched in antioxidants, and
b) providing behavioral enrichment to said animal:
wherein the feeding and providing behavioral enrichment combination is effective in increase neutrophil phagocytosis and B cell percentages in said animal.

2. The method of claim 1 wherein said behavioral enrichment comprises exercising the animal regularly effective to cause behavioral enrichment.

3. (canceled)

4. (canceled)

5. The method according to claim 1 wherein the diet comprises d1-alpha-lipoic acid at about 125 ppm or more.

6. (canceled)

7. (canceled)

8. The method according to claim 1, wherein the behavioral enrichment comprises hand walking of the animal for at least 30 minutes twice a week.

9-17. (canceled)

18. A method to enhance disease resistance in an animal comprising:
a) providing an animal feed composition enriched in vitamin E in an amount of about 1000 ppm to about 2000 ppm: and
b) providing instructions to enhance disease resistance in the animal fed the animal feed composition by providing behavioral enrichment to said animal;
wherein the combination of feeding the feed composition and providing behavioral enrichment to said animal is effective to increase neutrophil phagocytosis and B cell percentages in said animal.
